(19) United States
(12) Patent Application Publication
(10) Pub. No.: US 2018/0369391 A1
(43) Pub. Date: Dec. 27, 2018

(54) HYDROGEL FOAMS AND METHODS OF MAKING AND USING THE SAME

(71) Applicant: BioTime, Inc., Alameda, CA (US)

(72) Inventor: Michael V. Onorato, Oakland, CA (US)

(21) Appl. No.: 14/820,497

(22) Filed: Aug. 6, 2015

Related U.S. Application Data
(60) Provisional application No. 62/034,100, filed on Aug. 6, 2014.

(51) Int. Cl. A61K 47/36; A61K 9/70; C12N 5/0775; C08J 3/075; C08J 9/26

(52) U.S. Cl. CPC A61K 47/36; A61K 9/7007; C12N 5/0662; C08J 2305/08; C08J 9/26; C12N 2533/80; C08J 3/075

(57) ABSTRACT

The invention provides methods, compositions and kits relating to hydrogel foams.

### CROSS-REFERENCE TO RELATED APPLICATIONS

This application claims priority to U.S. Provisional Application No. 62/034,100, filed on Aug. 6, 2014, the entire contents of which are hereby incorporated by reference.

### FIELD OF THE INVENTION

The field of the invention relates to hyaluronate based hydrogel matrices such as hydrogel foams for in vivo and in vitro applications.

### BACKGROUND

Hydrogels are three dimensional hydrophilic, polymeric networks capable of imbibing large amounts of water or biological fluids. The networks may be comprised of homopolymers or copolymers (Peppas et al. (2000) European Journal of Pharmaceutics and Biopharmaceutics 50:27). While being highly hydrophilic, hydrogels are prevented from dissolving due to their chemically or physically cross-linked network. Water or biological fluids can penetrate between the polymer chains of the network causing swelling resulting in hydrogel formation. Hydrogels are appealing for biological applications because of their high water content and their biocompatibility (Peppas et al. (2006) Advanced Materials 18:1345). Synthetic hydrogels provide a delivery vehicle for a wide variety of therapeutics including large molecular weight protein and peptide drugs as well as cellular based therapeutics.

Hydrogels from many synthetic polymers such as poly (hydroxyethyl methacrylate) (PHEMA), poly-(ethylene glycol) (PEG) and poly (vinyl alcohol) (PVA) have been described (Peppas et al. (2006) Advanced Materials 18:1345). Hydrogels created from naturally sourced material such as collagen, hyaluronic acid (HA), fibrin, alginate, agarose and chitosan have also been described (Lee et al. (2001) Chem. Rev. 101:1869). Hyaluronate (HA) is a glycosaminoglycan that is comprised of repeating disaccharide units and is prevalent, for example, during wound healing and in joints.

Covalently cross-linked hydrogels formed by various chemical modifications have been described (Vercruysse et al. (1997) Bioconjugate Chem. 8:686; Prestwich et al. (1998) J. Controlled Release 53:93; Burdick et al. (2005) Biomacromolecules 6:386; Gamini et al. (2002) Biomaterials 23:1161; U.S. Pat. No. 7,928,069; U.S. Pat. No. 7,981,871).

Hydrogels based on thiol-modified derivatives of hyaluronic acid (HA) and porcine gelatin cross-linked with polyethylene glycol diacrylate (PEGDA) (trade name HyStem®) have unique chemical, biological and physical attributes making them suitable for many applications including cell culture, drug delivery and the like (Shu et al. (2004) J of Biomed Mat Res Part A 68:365; Shu et al. (2002) Biomacromolecules 3:1304; Vanderhooft et al. (2009) Macromolecular Biosci 9:20. Cross-linked HA hydrogels, including HyStem®, have been successfully used in animal models of corneal epithelial wound healing Yang et al. (2010) Veterinary Opthal 13:144, corneal tissue engineering (Espandar et al. (2012) Archives of Opthamol 130:202, and retinal repair Liu et al. (2013) Tissue Engineering Part A 19:135. Cross-linked HA hydrogels also provide a flexible platform, allowing a user to modulate both gel compliance and gelation time by adjusting the ratio of its components (Zheng et al. (2004) Biomaterials 25:1339; Vanderhooft et al. (2009) Macromolecular Bioscience 9:20). Since HyStem® gelation times are inversely proportional to final gel stiffness, higher concentrations of the PEGDA cross linker will cause HyStem to gel in approximately five minutes (G′>1300 Pa) while low concentrations require approximately one to two hours to form softer (G′<50 Pa) gels (Zheng et al. (2004) Biomaterials 25:1339; Vanderhooft et al. (2009) Macromolecular Bioscience 9:20; Hanjaya-Putra et al. (2010) J. Cell. And Molec. Med. 14:2436).

The preclinical use of hydrogels to maintain bioactivity and slow release of biologics has been described (Cai et al. (2005) Biomaterials 26:6054; Zhang (2011) Biomaterials 32:9415; Overman et al. (2012) Proceedings of the National Academy of Sciences of the United States of America 109:E2230; Garbern et al. (2011) Biomaterials 32:2407; Koutsopoulos et al. (2009) Proceedings of the National Academy of Sciences of the United States of America 106:4623. Furthermore, their use in cell delivery has been shown to improve cell viability and localization post-implantation (Laflamme et al. (2007) Nature Biotechnology 25:1015; Zhong et al. (2010) Neurorehabilitation and Neural Repair 24:636; Compte et al. (2009) Stem Cells 27:753. Several different hydrogels have been used as excipients in FDA-approved ocular small molecule therapeutics to increase their residence time on the eye surface (Kompella et al. (2010) Therapeutic Delivery 1:435.

In addition, two new hydrogel formulations have been reported which show promise in delivering therapeutic cells (Ballios et al. (2010) Biomaterials 31:2555; Caicco et al. (2012) Journal of Biomedical Materials Research Part A 101:1472; Yang et al. (2010) Veterinary ophthalmology 13:144; Mazumder et al. (2012) Journal of Biomedical Materials Research Part A 100:1877.

While previously described hydrogels, such as Hystem® have many desirable attributes and can be used in a wide variety of applications, it would, nonetheless, be useful to provide hydrogels with improved physical properties such as physical resilience to mechanical stress, increased tensile strength and a resistance to deformation. Additionally, it would be desirable to provide a hydrogel having a larger pore size to allow cellular infiltration.

Thus there is a need for improved hydrogels that provide improved biological, physical and chemical properties. There is also a need for improved methods of making hydrogels that provide for greater control of the physical and chemical characteristics of the hydrogel including, but not limited to, physical resilience, increased tensile strength, pore size, cytocompatibility and biocompatibility. Moreover, there is also a need to simplify the manufacture of hydrogels in a cost effective way. The invention described herein meets these needs as well as other needs in the field.

### SUMMARY OF THE INVENTION

In certain embodiments the invention provides hydrogel foams, methods of making hydrogel foams, and methods of using hydrogel foams.

In some embodiments the invention provides a hydrogel foam comprising hyaluronate. The hyaluronate may be thiolated.

In some embodiments the invention provides a hydrogel foam comprising gelatin. The gelatin may be thiolated.

In some embodiments the invention provides a hydrogel foam comprising polyethylene glycol diacrylate.

In some embodiments the invention provides a hydrogel foam comprising heparin. The heparin may be thiolated.

In other embodiments the invention provides a hydrogel foam comprising hyaluronate and gelatin. The hyaluronate may be thiolated. The gelatin may be thiolated.

In other embodiments the invention provides a hydrogel foam comprising hyaluronate, gelatin and polyethylene glycol diacrylate. The hyaluronate may be thiolated. The gelatin may be thiolated.

In still other embodiments the invention provides a hydrogel foam comprising hyaluronate, gelatin, heparin and polyethylene glycol diacrylate. The hyaluronate may be thiolated. The gelatin may be thiolated. The heparin may be thiolated.

In some embodiments the invention provides a hydrogel foam and at least one replicating cell, wherein the at least one replicating cell has infiltrated the interior of the hydrogel foam.

In some embodiments the invention provides a hydrogel foam comprising hyaluronate and at least one replicating cell, wherein the at least one replicating cell has infiltrated the interior of the hydrogel foam.

In some embodiments the invention provides a hydrogel foam comprising gelatin and at least one replicating cell, wherein the at least one replicating cell has infiltrated the interior of the hydrogel foam.

In some embodiments the invention provides a hydrogel foam comprising heparin and at least one replicating cell, wherein the at least one replicating cell has infiltrated the interior of the hydrogel foam.

In some embodiments the invention provides a hydrogel foam comprising polyethylene glycol diacrylate and at least one replicating cell, wherein the at least one replicating cell has infiltrated the interior of the hydrogel foam.

In other embodiments the invention provides a hydrogel foam comprising hyaluronate and gelatin and at least one replicating cell, wherein the at least one replicating cell has infiltrated the interior of the hydrogel foam.

In other embodiments the invention provides a hydrogel foam comprising hyaluronate, gelatin and polyethylene glycol diacrylate and at least one replicating cell, wherein the at least one replicating cell has infiltrated the interior of the hydrogel foam.

In other embodiments the invention provides a hydrogel foam comprising hyaluronate, gelatin, heparin and polyethylene glycol diacrylate and at least one replicating cell, wherein the at least one replicating cell has infiltrated the interior of the hydrogel foam.

In still other embodiments the invention provides a conditioned hydrogel.

In further embodiments the invention provides a conditioned hydrogel comprising hyaluronate. The hyaluronate may be thiolated.

In yet other embodiments the invention provides a conditioned hydrogel comprising gelatin. The gelatin may be thiolated.

In still other embodiments the invention provides a conditioned hydrogel comprising heparin. The heparin may be thiolated.

In some embodiments the invention provides a conditioned hydrogel comprising polyethylene glycol diacrylate.

In certain embodiments the invention provides a conditioned hydrogel comprising hyaluronate and polyethylene glycol diacrylate. The hyaluronate may be thiolated.

In still further embodiments the invention provides a conditioned hydrogel comprising hyaluronate, gelatin and polyethylene glycol diacrylate. The hyaluronate may be thiolated. The gelatin may be thiolated.

In still further embodiments the invention provides a conditioned hydrogel comprising hyaluronate, gelatin, heparin and polyethylene glycol diacrylate. The hyaluronate may be thiolated. The geltin may be thiolated. The heparin may be thiolated.

In further embodiments the invention provides a method of making a hydrogel foam comprising a) combining at least one polymer and one cross-linking agent in a device comprising at least one pore b) applying an external force to the device from step a) such that the at least one polymer and at least one cross-linking agent pass through the pore while allowing the at least one polymer and at least one cross-linking agent to polymerize and form a hydrogel; c) casting the hydrogel from step b) into a vessel; d) freezing the cast hydrogel from step c); e) lyophilizing the frozen hydrogel from step d); f) optionally compressing the lyophilized hydrogel (the hydrogel foam) from step e) by applying an external force to the lyophilized hydrogel from step e) thereby forming a compressed hydrogel foam.

In yet other embodiments the invention provides a method of making a hydrogel foam comprising a) combining hyaluronate and polyethylene glycol diacrylate in a device comprising at least one pore b) applying an external force to the device from step a) such that the hyaluronate and polyethylene glycol diacrylate pass through the pore while allowing the hyaluronate and polyethylene glycol diacrylate to polymerize and form a hydrogel; c) casting the hydrogel from step b) into a vessel; d) freezing the cast hydrogel from step c); e) lyophilizing the frozen hydrogel from step d); f) optionally compressing the lyophilized hydrogel from step e) by applying an external force to the lyophilized hydrogel (the hydrogel foam) thereby forming a compressed hydrogel foam.

In certain embodiments the invention provides a method of making a hydrogel foam comprising a) combining gelatin and polyethylene glycol diacrylate in a device comprising at least one pore b) applying an external force to the device from step a) such that the gelatin and polyethylene glycol diacrylate pass through the pore while allowing the gelatin and polyethylene glycol diacrylate to polymerize and form a hydrogel; c) casting the hydrogel from step b) into a vessel; d) freezing the cast hydrogel from step c; e) lyophilizing the frozen hydrogel from step d); f) optionally compressing the lyophilized hydrogel (the hydrogel foam) from step e) by applying an external force to the lyophilized hydrogel thereby forming a compressed hydrogel foam.

In yet other embodiments the invention provides a method of making a hydrogel foam comprising a) combining hyaluronate, gelatin and polyethylene glycol diacrylate in a device comprising at least one pore b) applying an external force to the device from step a) such that the hyaluronate, gelatin and polyethylene glycol diacrylate pass through the pore while allowing the hyaluronate, gelatin and polyethylene glycol diacrylate to polymerize and form a hydrogel; c) casting the hydrogel from step b) into a vessel; d) freezing the cast hydrogel from step c) lyophilizing the frozen hydrogel from step d); compressing the lyophilized hydrogel (the hydrogel foam) from step d) by applying an external force to the lyophilized hydrogel thereby forming a compressed hydrogel foam.

In further embodiments the invention provides a method of conditioning a hydrogel comprising a) combining at least one polymer and one cross-linking agent in a device comprising at least one pore b) applying an external force to the device from step a) such that the at least one polymer and at least one cross-linking agent pass through the pore while allowing the at least one polymer and at least one cross-linking agent to polymerize and form a hydrogel thereby conditioning a hydrogel.

In other embodiments the invention provides a method of conditioning a hydrogel comprising a) combining hyaluronate and polyethylene glycol diacrylate in a device comprising at least one pore b) applying an external force to the device from step a) such that the hyaluronate and polyethylene glycol diacrylate pass through the pore while allowing the hyaluronate and polyethylene glycol diacrylate to polymerize and form a hydrogel thereby conditioning a hydrogel.

In still other embodiments the invention provides a method of conditioning a hydrogel comprising a) combining gelatin and polyethylene glycol diacrylate in a device comprising at least one pore b) applying an external force to the device from step a) such that the gelatin and polyethylene glycol diacrylate pass through the pore while allowing the gelatin and polyethylene glycol diacrylate to polymerize and form a hydrogel thereby conditioning a hydrogel.

In further embodiments the invention provides a method of conditioning a hydrogel comprising a) combining hyaluronate, gelatin and polyethylene glycol diacrylate in a device comprising at least one pore b) applying an external force to the device from step a) such that the hyaluronate, gelatin and polyethylene glycol diacrylate pass through the pore while allowing the hyaluronate, gelatin and polyethylene glycol diacrylate to polymerize and form a hydrogel thereby conditioning a hydrogel.

In still further embodiments the invention provides a method of conditioning a hydrogel comprising a) combining hyaluronate, gelatin, heparin and polyethylene glycol diacrylate in a device comprising at least one pore b) applying an external force to the device from step a) such that the hyaluronate, gelatin, heparin and polyethylene glycol diacrylate pass through the pore while allowing the hyaluronate, gelatin, heparin and polyethylene glycol diacrylate to polymerize and form a hydrogel thereby conditioning a hydrogel.

In some embodiments the invention provides a method of making a hydrogel foam comprising conditioning a hydrogel according to any one of the methods of conditioning a hydrogel described infra.

In other embodiments the invention provides a method of treating a subject in need of treatment for a disease or a condition comprising administering a hydrogel foam to the subject. The hydrogel foam may comprise one or more therapeutic agents.

In certain embodiments the invention provides a kit comprising at least one polymer, at least one cross linking agent, and at least one device comprising one or more pores.

In certain embodiments the invention provides a peptide-functionalized polymer. The peptide may comprise the amino acid sequence RGD.

In some embodiments the invention provides a peptide-functionalized carboxy methylated thiolated HA (CMHA-S).

In still other embodiments the invention provides a peptide-functionalized CMHA-S wherein the peptide comprises RGD.

In yet other embodiments the invention provides an RGD peptide-functionalized CMHA-S.

In further embodiments the invention provides a hydrogel comprising a polymer functionalized with a peptide.

In some embodiments the invention provides a hydrogel functionalized with a peptide comprising RGD.

In yet other embodiments the invention provides a hydrogel comprising CMHA-S functionalized with a peptide.

In still further embodiments the invention provides a hydrogel comprising CMHA-S functionalized with a peptide comprising RGD.

In yet other embodiments the invention provides a hydrogel comprising CMHA-S functionalized with an RGD peptide.

In further embodiments the invention provides a composition comprising a polymer functionalized with a peptide and a cell. The cell may be attached to the polymer functionalized with the peptide.

In some embodiments the invention provides a composition comprising CMHA-S functionalized with a peptide and a cell. The cell may be attached to the CMHA-S functionalized with the peptide.

In other embodiments the invention provides a composition comprising CMHA-S functionalized with a peptide comprising RGD and a cell. The cell may be attached to the CMHA-S functionalized with a peptide comprising RGD.

In still other embodiments the invention provides a composition comprising CMHA-S functionalized with an RGD peptide and a cell. The cell may be attached to the CMHA-S functionalized with an RGD peptide.

The functionalized monomers and hydrogels described above may be used to make hydrogel foams described infra.

The hydrogel foams described infra may be comprised of any of the functionalized monomers or hydrogels described above.

### DESCRIPTION OF DRAWINGS

For a fuller understanding of the nature and advantages of the present invention, reference should be had to the following detailed description taken in connection with the accompanying drawings, in which:

FIG. 1 is a photograph showing a conditioned hydrogel in a syringe.

FIG. 2 is a photograph of a conditioned hydrogel after casting in a mold.

FIG. 3A is a photograph showing a conditioned lyophilized hydrogel i.e. a hydrogel foam before compression; FIG. 3B is a photograph showing a hydrogel foam after moderate compression; FIG. 3C is a photograph showing a hydrogel foam completely compressed.

FIG. 4 shows two formulations of compressed hydrogel foams that have been cast, lyophilized, compressed, and rehydrated as described infra. Formulation α (top row): 100 mg thiol-modified hyaluronate, 100 mg thiol-modified gelatin, 200 mg PEGDA (1:1:2 w/w) in 25 ml deionized, degassed (DG) water. Formulation β (bottom row): 100 mg thiol-modified hyaluronate, 100 mg thiol-modified gelatin, 50 mg PEGDA (2:2:1 w/w) in 25 ml DG water.

FIGS. 5A-5C depict culture of human mesenchymal stem cells (hMSC) on hydrogel foams. FIG. 5A is a photomicrograph that shows a culture of hMSC on the β formulation of the hydrogel foam 3 days after seeding; cells are viable as evidenced by Calcein AM staining. The image shows the edge of the hydrogel foam. FIG. 5B is a photomicrograph that shows a culture of hMSC on the α formulation of the hydrogel foam 3 days after seeding; cells are viable as evidenced by Calcein AM staining. The image shows the edge of the hydrogel foam. FIG. 5C is a photomicrograph that shows a culture of hMSC on the α formulation of the hydrogel foam 3 days after seeding; cells are viable as evidenced by Calcein AM staining. The image shows the middle of the hydrogel foam. Magnification of all images was 4×.

FIGS. 6A-6B depict culture of human mesenchymal stem cells (hMSC) on hydrogel foams. FIG. 6A is a photomicrograph showing hMSC growing 10 days after seeding on the β formulation of the hydrogel foam. The image shows the edge of the hydrogel foam. FIG. 6B is a photomicrograph showing hMSC growing 10 days after seeding on the α formulation of the hydrogel foam. The image shows the middle of the hydrogel foam. Magnification of all images was 4×.

FIG. 7 is a photomicrograph showing hMSCs growing 10 days after seeding on the α formulation of the hydrogel foam. The image is of the edge of the hydrogel foam and the cells are stained with both Calcein AM and propidium iodide. Magnification was 4×.

FIG. 8 is a photograph of a large volume gel foam cast in 100 mm² petri dish and cut into a square after removal from the mold. Gel foam technique described in the instant application is not limited to small volumes and sizes; gel foams can be cut into desired shapes and multiple sections post-production.

FIG. 9 is a photograph of a gel foam after hydration in cell culture media. Gel foams remain intact and retain their shape after hydration and and can be robustly handled in both hydrated and non-hydrated states.

FIGS. 10A-10B are photomicrographs showing hMSCs cultured on a gel foam for 3 days (FIG. 10A) and 9 days (FIG. 10B) stained with hematoxylin and eosin. The majority of the cells remain on the surface, with minor infiltration into the foam interior. Where a breach is observed, cell infiltration into the interior of the gel foam occurs.

FIG. 11 is a photograph showing the casting of gel foams into different three dimensional shapes. The gel foams retain their shape after lyphilization.

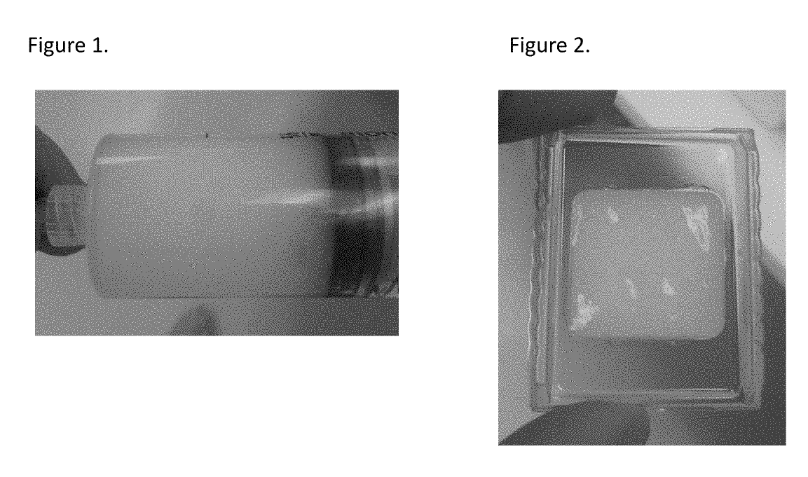

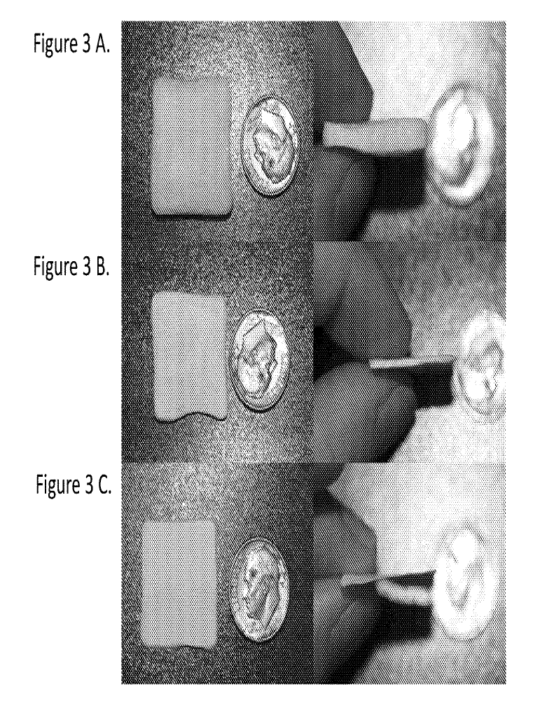

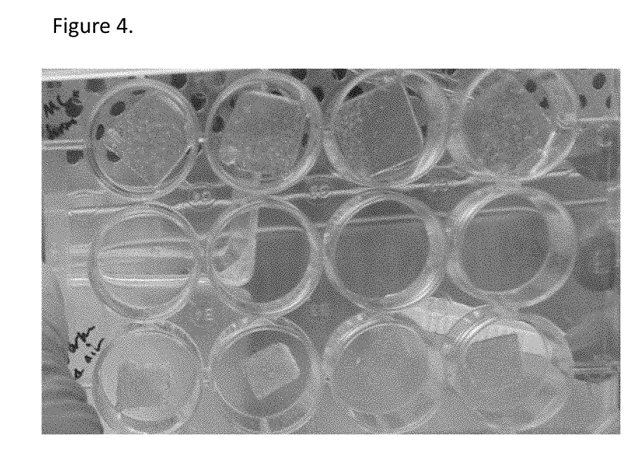

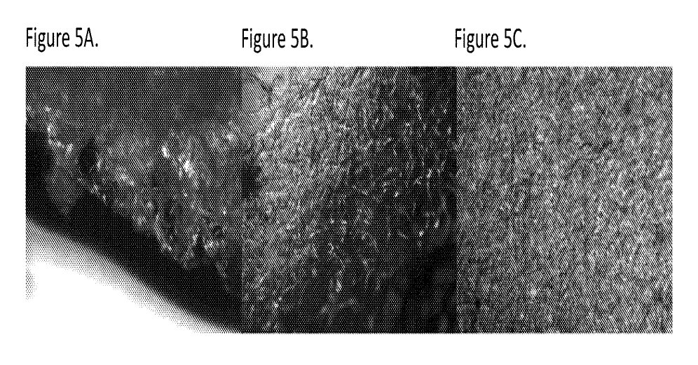

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Any methods and materials similar or equivalent to those described herein can be used in the practice or testing of embodiments of the present disclosure.

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 10% of the numerical value of the number with which it is being used. Therefore, about 50% means in the range of 40% to 60%.

The term “antibody”, as used herein, means an immunoglobulin or a part thereof, and encompasses any polypeptide comprising an antigen-binding site regardless of the source, method of production, or other characteristics. The term includes for example, polyclonal, monoclonal, monospecific, polyspecific, humanized, single-chain, chimeric, synthetic, recombinant, hybrid, mutated, and CDR-grafted antibodies. A part of an antibody can include any fragment which can bind antigen, for example, an Fab, F(ab′)₂, Fv, scFv.

The term “carboxymethylated, thiolated hyaluronic acid,” as used herein, refers to hyaluronan modified with both a carboxymethyl group and a thiol group (see U.S. Pat. Nos. 7,928,069 and 7,981,871).

The term “compression or compressed,” as used herein refers to the application of balanced inward (“pushing”) forces to different points on a material or structure, that is, forces with no net sum or torque directed so as to reduce its size in one or more directions.

The “conditioned hydrogel,” as used herein, refers to a hydrogel that has had its macrostructure disrupted by the application of an external force, such as a mechanical force.

The term “crosslinking agent,” as used herein, refers to an agent such as a multifunctional, e.g., difunctional substance, that promotes covalent bonding between two or more monomers to form a polymer by reacting with reactive groups on the monomers. Other examples of crosslinking agents include oxidizing agents which can cause monomers, such as thiolated monomers to react with each other. Examples of a crosslinking agent is polyethylene glycol diacrylate (PEGDA), hydrogen peroxide, and glutathione disulfide (GSSG).

The term “hyaluronate” “hyaluronic acid” “HA,” as used herein, refers to a polymer of disaccharides, composed of D-glucuronic acid and D-N-acetylglucosamine, linked via alternating β-1,4 and β-1,3 glycosidic bonds. It is an anionic, nonsulfated glycosaminoglycan. Hyaluronate can be any length or size. Polymers of hyaluronate can range in size from 5,000 to 20,000,000 Da in vivo.

The term “hydrogel,” as used herein, refers to a hydrophilic polymer.

The term “hydrogel foam”, as used herein, refers to a hydrogel that is conditioned and lyophilized. In some embodiments, the hydrogel foam is compressed by applying external force to the hudrogel foam after lyphilization.

The term “monomer” or “macromonomer” as used herein refers to a chemical moiety that under appropriate conditions can polymerize with itself and/or another moiety to form a polymer. Examples of monomers include HA, CMHA, gelatin and the like. In certain instances the monomer unit can itself be a polymer, e.g. HA is polymer of two disaccharides that can undergo further polymerization.

The use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A “polynucleotide” or “oligonucleotide” may comprise DNA, RNA, cDNA, PNA or a polymer of nucleotides linked by phosphodiester and/or any alternate bonds.

The term “peptide,” as used herein, refers to two or more amino acids joined by a peptide bond. A peptide can, in some instances, be a portion of a full length protein.

The term “polymer,” as used herein, refers to any of a class of natural or synthetic substances composed of macromolecules that are multiples of monomers. The monomers need not all be the same or have the same structure. Polymers may consist of long chains of unbranched or branched monomers or may be cross-linked networks of monomers in two or three dimensions. Their backbones may be flexible or rigid. Some natural inorganic materials (e.g., the minerals diamond, graphite, and feldspar) and certain man-made inorganic materials (e.g., glass) have polymer-like structures. Many important natural materials are organic polymers, including cellulose (from sugar monomers; polysaccharides), hyaluronan, lignin, rubber. Synthetic organic polymers include many plastics, including polyethylene, the nylons, polyurethanes, polyesters, vinyls (e.g., PVC), and synthetic rubbers.

By “pharmaceutically acceptable”, it is meant the carrier, diluent or excipient must be compatible with the other ingredients of the formulation and not deleterious to the recipient thereof.

The term “subject,” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” refers to a male. In some embodiments, the term “subject,” refers to a female.

The term “suitable media,” as used herein, refers to a solution that can be used to grow cells in culture. A suitable media may include a formulation of salts and/or buffering reagents. A suitable media may include any or all of the following: salts, sugars, amino acids, proteins, growth factors, cytokines, and hormones, additives such as serum, albumin, antibiotics, insulin, selenium and transferrin. Suitable culture media includes for example commercially available culture media such as DMEM, MEM, Stem Pro and the like.

“Therapeutic Agent,” as used herein, refers to any agent administered to a subject to treat a condition, including but not limited to a molecule such as a small molecule, a moiety, a peptide, a protein, a lipid, a polysaccharide, a nucleic acid, an antibody, a cell, a tissue or organ, a hormone, a growth factor, a cytokine, a drug.

A “therapeutically effective amount” of a composition such as a therapeutic agent is a predetermined amount calculated to achieve the desired effect. In some embodiments, the effective amount is a prophylactic amount. In some embodiments, the effective amount is an amount used to medically treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable excipient composition, it is sufficient to achieve an effective systemic concentration or local concentration in the targeted tissue.

The term “Thiolated hyaluronate” refers to hyaluronate modified with a thiol group (see U.S. Pat. Nos. 7,928,069 and 7,981,871).

The term “Thiolated Gelatin” or “Thiolated Porcine Gelatin,” as used herein refers, to a gelatin molecule functionalized with a thiol group (see U.S. Pat. Nos. 7,928,069 and 7,981,871).

The terms “treat,” “treated,” or “treating,” as used herein, can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results may include, but are not limited to one or more of the following: alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response. Treatment also includes prolonging survival as compared to expected survival if not receiving treatment.

The term “tissue” refers to any aggregation of similarly specialized cells that are united in the performance of a particular function.

The term “thiol” as used herein, refers to an organosulfur compound that contains a carbon-bonded sulfhydryl (—C—SH or R—SH) group (where R represents an alkane, alkene, or other carbon-containing group of atoms). A “thiolated molecule,” as used herein, can refer to either the reduced (SH) or oxidized (S—S) form of the group.

In certain embodiments the invention provides a hydrogel foam. By conditioning a hydrogel foam, as described infra, a hydrogel foam is obtained with improved physical properties. The hydrogel foam is for example, more durable then previously described hydrogel foams. The hydrogel foam of the invention can be stored and shipped frozen thereby facilitating commercial distribution.

### Hydrogel Foams

In some embodiments the invention provides a hydrogel foam. The hydrogel foam may be comprised of one or more monomers cross-linked to form a polymer, e.g. a polymer hydrogel (see e.g. U.S. Pat. Nos. 7,981,871; 7,928,069). The monomers may be a naturally occurring monomer e.g. hyaluronate, gelatin. The monomers may be cross-linked to form the polymer hydrogel using a cross linking agent. One suitable cross linking agent is polyethylene glycol diacrylate (PEGDA). In some embodiments the polymer hydrogel may further comprise heparin. The heparin may allow therapeutic agents to be bound to the polymer hydrogel.

The monomers comprising the polymer hydrogel may be thiolated e.g. derivatized with one or more thiol groups. Exemplary thiolated monomers include thiolated hyaluronate, thiolated gelatin, and thiolated heparin. In some instances the monomer may be a carboxymethylated thiol monomer, e.g. carboxy-methyl thiolated hyaluronate, carboxy-methyl thiolated gelatin, carboxy-methylated heparin.

In some embodiments the polymer hydrogels comprise thiolated hyaluronate cross-linked with PEGDA. In some embodiments the polymer hydrogels comprise thiolated hyaluronate and thiolated gelatin cross-linked with PEGDA. In some embodiments the polymer hydrogels comprise thiolated hyaluronate, thiolated gelatin, and thiolated heparin cross-linked with PEGDA.

In certain embodiments one or more of the monomers comprising the hydrogel may be functionalized with one or more molecules. Examples of suitable molecules include, proteins, peptides, nucleic acids, lipids and polysaccharides. In some embodiments the monomer may be functionalized with a peptide. The peptide may contain an RGD (arginine, glycine aspartic acid) sequence known to facilitate cell attachment. Suitable monomers include hyaluronate, such as thiolated hyaluronate including carboxymethyl hyaluronate; gelatin such as thiolated gelatin and heparin such as thiolated heparin.

Hydrogel foams may be made from hydrogels by conditioning, freezing and casting the hydrogels as described in detail below. The hydrogel foams provide a denser, more malleable, stronger, smaller, polymer material relative to a hydrogel that is not a hydrogel foam (i.e. a hydrogel that has been polymerized and cast but not conditioned, frozen and lyophilized). The hydrogel foam may have larger relative pore size compared to a hydrogel that is not a hydrogel foam.

The hydrogel foam may have a thickness ranging from about 0.01 mm-10 mm, from about 0.05 mm-5 mm; from about 0.1 mm-5 mm; from about 0.2 mm-4 mm; from about 0.3 mm-3.5 mm. The hydrogel foam may have a thickness before it is compressed of about: 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm 18 mm, 19 mm, 20 mm. The hydrogel foam may have a thickness after compression of about: 0.01 mm, 0.05 mm 0.08 mm; 0.1 mm, 2 mm, 0.3 mm, 0.4 mm 0.5 mm, 0.6 mm, 0.7 mm, 0.8 mm, 0.9 mm 1.0 mm; 1.2 mm, 1.3 mm, 1.4 mm, 1.5 mm 1.6 mm 1.7 mm, 1.8 mm; 1.9 mm, 2.0 mm, 2.5 mm, 3.0 mm, 3.5 mm, 4.0 mm, 4.5 mm 5.0 mm. In one embodiment the uncompressed hydrogel foam has a thickness of about 3.5 mm. In one embodiment the compressed hydrogel has a thickness of about 1.3 mm. In one embodiment the compressed hydrogel has a thickness of about 0.38 mm. In one embodiment the compressed hydrogel has a thickness of about 0.16 mm. In one embodiment the compressed hydrogel has thickness ranging from about 0.16 mm-0.38 mm.

Those skilled in the art will appreciate that hydrogel foams may be cast in various sizes and thicknesses and are not limited to small sizes or volumes. Large volume gels (which may also be thicker than those described above) may be cut into desired shapes and multiple sections post production.

The hydrogel foams of the invention typically will be dehydrated after lyophilization as described below. The hydrogel foam may be rehydrated by placing the hydrogel foam in a suitable liquid. Suitable liquids may include water, an isotonic solution, a buffer such as PBS or hepes, a suitable media, including any commercially available cell culture media, e.g., DMEM, Media 199, StemPro, M-TESR, RPMI, Hams F12 and the like.

Rehydrated hydrogel foams may retain from about 1 gram of solution/gram of hydrogel foam to about 30 grams of solution/gram of hydrogel foam; from about 2 grams of solution/gram of hydrogel foam to about 20 grams of solution/gram of hydrogel foam; from about 5 grams of solution/gram of hydrogel foam to about 15 grams of solution/gram of hydrogel foam; from about 6 grams of solution/gram of hydrogel foam to about 12 grams of solution/gram of hydrogel foam. In some embodiments of the invention the hydrogel foam retains about 5 grams of solution/gram of hydrogel foam; about 6 grams of solution/gram of hydrogel foam; about 7 grams of solution/gram of hydrogel foam; about 8 grams of solution/gram of hydrogel foam; about 9 grams of solution/gram of hydrogel foam; about 10 grams of solution/gram of hydrogel foam; about 11 grams of solution/gram of hydrogel foam; about 12 grams of solution/gram of hydrogel foam; about 13 grams of solution/gram of hydrogel foam; about 14 grams of solution/gram of hydrogel; foam about 15 grams of solution/gram of hydrogel foam.

In some embodiments of the invention the hydrogel foam of the invention retains about 1 gram of solution/100 cm² of hydrogel foam-about 50 grams of solution/100 cm² of hydrogel foam about 2 gram of solution/100 cm² of hydrogel foam-about 25 grams of solution/100 cm² of hydrogel foam; about 4 gram of solution/100 cm² of hydrogel foam-about 30 grams of solution/100 cm² of hydrogel foam; about 5 gram of solution/100 cm² of hydrogel foam-about 25 grams of solution/100 cm² of hydrogel foam; about 6 gram of solution/100 cm² of hydrogel foam-about 20 grams of solution/100 cm² of hydrogel foam; about 7 gram of solution/100 cm² of hydrogel foam-about 15 grams of solution/100 cm² of hydrogel foam. In some embodiments of the invention the hydrogel foam retains about 1 gram of solution/100 cm² of hydrogel foam; about 5 grams of solution/100 cm² of hydrogel foam; about 6 grams of solution/100 cm² of hydrogel foam; about 7 grams of solution/100 cm² of hydrogel foam; about 8 grams of solution/100 cm² of hydrogel foam; about 9 grams of solution/100 cm² of hydrogel foam; about 10 grams of solution/100 cm² of hydrogel foam; about 11 grams of solution/100 cm² of hydrogel foam; about 12 grams of solution/100 cm² of hydrogel foam; about 13 grams of solution/100 cm² of hydrogel foam; about 14 grams of solution/100 cm² of hydrogel foam; about 15 grams of solution/100 cm² of hydrogel foam; about 16 grams of solution/100 cm² of hydrogel foam; about 17 grams of solution/100 cm² of hydrogel foam; about 18 grams of solution/100 cm² of hydrogel foam; about 19 grams of solution/100 cm² of hydrogel foam; about 20 grams of solution/100 cm² of hydrogel foam.

### Methods of Making Hydrogel Foams

In certain embodiments the invention provides methods of making hydrogel foams. In some embodiments the invention provides a method of making a hydrogel foam comprising a) combining one or more monomers with at least one cross-linking agent b) conditioning the combination from step a); c) freezing the conditioned combination from step b) and d) lyophilizing the frozen combination from step c thereby making a hydrogel foam. The conditioned combination of step b) may be cast into a suitable vessel. Optionally the lyophilized sponge may be compressed to a desired thickness.

Methods of making hydrogels have been described, see U.S. Pat. Nos. 7,981,871 and 7,928,069. Suitable monomers include hyaluronate, gelatin, and heparin. The monomers may be thiolated. The monomers may be carboxymethylated. The monomers may be both thiolated and carboxymethylated. Suitable crosslinking agents include acrylates such as polyethylene glycol dimethylacrylate, oxidizing agents such as hydrogen peroxide and GSSG.

In some embodiments of the invention the hydrogel is comprised of one monomer and at least one cross linking agent. In some embodiments of the invention the hydrogel is comprised of two different monomers and at least one cross linking agent. In some embodiments of the invention the hydrogel is comprised of 3 monomers and at least one cross linking agent. In some embodiments of the invention the hydrogel is comprised of 3 or more monomers and at least one cross linking agent. The monomers may be combined at a temperature ranging from about 1° C.-30° C.; 5° C.-25° C.; 10° C.-20° C. In some embodiments the monomers are combined at room temperature.

A skilled artisan will appreciate that the concentration of any of the monomers may be varied depending on the desired properties of the hydrogel.

Once the monomer(s) have been combined with at least one cross linking agent, the mixture may be conditioned. The mixture may polymerize to form a hydrogel either, before, after or simultaneously with conditioning. Conditioning may include passaging the mixture, by the application of an external force, through a device or material comprising at least one opening such as a pore, or application of an external force as to disrupt material macrostructure. Suitable devices or materials comprising at least one opening such as a pore include a syringe, a pipette, a membrane, a sieve, mixers (both overhead and in-line) and the like. Any external force may be used to cause the material to pass through the device or material comprising at least one pore. Examples of suitable external forces include a force applied by the human hand, the force applied by a mechanical pump. The force may be a vacuum, a centrifugal force and the like. Application of an external force to disrupt material macrostructure may include, but not limited to, shear forces as applied by a mixer (in line or stirred vessel), agitation of material, or addition of a species as to disrupt macrostructure. In some embodiments the mixture may be passaged through the device or material comprising at least one pore at least once. In other embodiments the mixture may be passaged through the device at least 1-1,000 times, at least 10-500 times, at least 20-300 times, at least 30-200 times, at least 40-100 times. In some embodiments the mixture is passed through the material or device comprising at least pore about 1 time, about 5 times, about 10 times, about 20 times, about 30 times, about 40 times, about 50 times, about 60 times, about 70 times, about 80 times, about 90 times, about 100 times. In some embodiments of the invention the mixture may be passed through the device or material comprising at least one pore more than 100 times. Conditioning may be performed at a temperature ranging from about 1° C.-30° C.; 5° C.-25° C.; 10° C.-20° C. In some embodiments conditioning is performed at room temperature.

After conditioning the hydrogel may be cast in a suitable vessel. Any vessel capable of holding the mixture may be used. Suitable vessels include a syringe, a multiwell plate, a tissue culture flask, a petri dish, a microscope slide, a tube, such as centrifuge tube, a three dimensional cast and the like.

After conditioning, and in some instances after casting, the hydrogel may be frozen. The hydrogel may be exposed to a constant temperature or a temperature gradient in order to freeze it. The hydrogel may be frozen below its triple point in order to facilitate lyophilization. In some embodiments the conditioned hydrogel may be frozen at about a temperature ranging from about 0° C.-−210° C., about −10° C.-−196° C., about −20° C.-−80° C. In some embodiments the conditioned hydrogel is frozen at about 0° C., about −10° C., about −20° C., about −50° C., about −100° C., about −196° C., about −210° C.

The conditioned hydrogel may be frozen for about one-24 hours, about 2-20 hours, about 3-10 hours, about 5-6 hours. In some embodiments of the invention the conditioned hydrogel may be frozen for about 1 day, 2 days, 3 days, 4 days or more. In some embodiments the conditioned hydrogel may be frozen for about 5 minutes or less, 10 minutes or less, 30 minutes or less, 60 minutes or less, 120 minutes or less, 240 minutes or less. In some embodiments of the invention the conditioned hydrogel may be frozen for about 1 hour, 2 hours, 2 hours, 4 hours, 5 hours, 6 hours, 7 hours, 8 hours, 9 hours 10 hours, 11 hours, 12 hours.

After the conditioned hydrogel has been frozen it may be lyophilized. Any lyophilizer may be used, e.g. a tray-style lyophilizer Lyophilization is performed under conditions of low pressure/high vacuum. Lyophilization may be conducted at a temperature of about −30° C.-−100° C., −50° C.-−80° C., −60-−70° C. In some embodiments lyophilization is performed at a temperature of about −30° C., −40° C., −50° C., −60° C., −70° C., −80° C. In some embodiments lyophilization is performed at about −51° C.

Lyophilization is performed under conditions of low pressure/high vacuum. In some embodiments lyophilization is performed at a pressure of about 0.010 bar-0.3 bar, about 0.02 bar-0.1 bar, about 0.03 bar-0.09 bar, 0.04 bar-0.07 bar. In some embodiments of the invention lyophilization is performed at a pressure of about 0.01 bar, 0.02 bar, 0.03 bar, 0.04 bar. 0.05 bar, 0.06 bar, 0.07 bar, 0.08 bar, 0.08 bar, 0.09 bar, 0.10 bar.

Lyophilization is performed at extended times to ensure acceptable water content of material is obtained. Lyophilization is performed for about 2-96 hours, 10-72 hours, 24-48 hours. In some embodiments of the invention, lyophilization is performed for 2 hours, for 4 hours, for 8 hours, for 12, hours, for 24 hours, for 48 hours, for 72 hours, and for 94 hours.

After lyophilization the hydrogel foam may be compressed. Compression may be achieved by placing the hydrogel foam under a pressure from an external force. For example, the hydrogel foam may be placed between two plates or two rollers and an external force may be applied. In one example, the human hand may be used to apply an external force to one or both of the plates that sandwich the hydrogel foam. The external force may be supplied by applying a weight to one of the plates. The external force may be supplied by a mechanical device. This applied external force may range from 0.1 psi to 1000 psi

### Culturing Cells on Hydrogel Foams

The hydrogel foams described infra may be used for culturing cells in vitro. Once cultured the cells may be implanted in a subject or used as research tools. To culture cells on the hydrogel foams described infra the hydrogel foam may be sterilized to prevent contamination of the cells. Sterilization may be performed by exposing the hydrogel foam to UV light. Sterilization may be performed by exposing the hydrogel to Ethylene oxide. Sterilization may be performed by exposing the hydrogel to gamma radiation or electron beam. Sterilization may be performed by washing the hydrogel foams one or more times in a 70% solution of ethanol followed by at least one wash with a neutral buffer, e.g. PBS or a commercially available cell culture media. In certain embodiments the hydrogel may be washed 3 or more times with the neutral buffer or the commercially available cell culture media.

Cells may be seeded on the hydrogel foams by contacting the hydrogel foam with a solution containing the cells. The solution may comprise a suitable media such as a commercially available cell culture media. The media may be supplemented with serum such as fetal calf serum. The media may be supplemented with a serum substitute such as knock out serum replacement (Life Technologies) or a solution comprising insulin, selenium and transferring. The solution of the cells and the foam are incubated for a sufficient time to allow the cells to attach to the hydrogel foam. Typically the cells and the hydrogel foam are incubated at 37° C., 5% CO₂. The cells may be grown in a suitable media under the same conditions until they reach confluency at which time the culture may be split by removing the cells from the hydrogel foam and reseeding a portion of the culture on a new hydrogel foam. Cells may be removed from the hydrogel by treating the cells with a detachment reagent such as trypsin. The cells may also be removed from the hydrogel foam mechanically. Alternatively, the cells are not removed from the hydrogel foam and the confluent cells are used for one of the applications described infra.

Cells suitable for growth on the hydrogel foams described infra include eukaryotic cells such as mammalian cells. Mammalian cells may include cells of human, non-human primate, murine, rat, hamster, guinea pig, cow, sheep, pig, horse origin, to name but a few. The cell may be stem cell, such as a pluripotent stem cell or a multipotent stem cell. Suitable stem cells may include an adult stem cell, an embryonic stem cell, an induced pluripotent stem cell and a stem cell obtained by nuclear transfer. The cell may be a primary cell isolated from a subject. The cell may be an established cell line, such as an immortalized cell line.

### Hydrogel Foam Applications and Uses

The hydrogel foams described infra may be used in vitro and in vivo. In vitro uses include research applications including in vitro cell culture, drug discovery and toxicity study. For example the hydrogel foams may be used to study the kinetics of drug release. The hydrogel foams may be used to study the effects of drug release over time on the metabolic activity of a test cell or tissue. The hydrogel foams described infra may be used to study cell growth, cell viability, and cell morphology in vitro.

The hydrogel foams may be used to grow cells in vitro. The cells themselves may be harvested from the hydrogel foams and used in research or therapeutically e.g., cell transplant or implant. Alternatively, one or more molecules produced by the cells grown on the hydrogel foams of the invention may be harvested and used in research or therapy. Examples of molecules produced by cells grown on the hydrogel foams of the invention may include, proteins, such as cytokines, growth factors, hormones and the like, antibodies, peptides, nucleic acids, such as DNA, mRNA, miRNA and the like, lipids, polysaccharides.

The hydrogel foams may be administered or implanted into a subject in need of treatment of disease or a condition. The hydrogel foams may be administered to a subject either alone or in combination with a therapeutic agent. When administered with a therapeutic agent the hydrogel foam and the therapeutic agent may be covalently linked, non-covalently linked (e.g. bonded by ionic interactions; hydrogen bonded, van der Waals forces) or encased within the hydrogel foam.

Examples of therapeutic agents include small molecules, biologics, peptides, proteins, nucleic acids (including cDNA, RNA, siRNA, PNA and the like). Therapeutic agents may include, but are not limited to antibiotics, anti-virals, anti-cancer drugs, growth factors, hormones, cytokines, anti-inflammatory drugs, nervous system modulators, pain relievers, narcotics and antibodies.

The hydrogel foams of the invention may be used as a depot to control the release of a therapeutic agent over time. The hydrogel foam can be implanted into a subject such that the therapeutic agent is delivered directly to the site where therapy is needed. Examples of therapeutic agents that can be delivered using the hydrogel foams of the invention include anti-cancer agents, anti-inflammatory agents such as, but not limited to, pilocarpine, hydrocortisone, prednisolone, cortisone, diclofenac sodium, indomethacin, 6.varies.-methyl-prednisolone, corticosterone, dexamethasone, prednisone, and the like; antibacterial agents including, but not limited to, penicillin, cephalosporins, bacitracin, tetracycline, doxycycline, gentamycin, chloroquine, vidarabine, and the like; analgesic agents including, but not limited to, salicylic acid, acetaminophen, ibuprofen, naproxen, piroxicam, flurbiprofen, morphine, and the like; local anesthetics including, but not limited to, cocaine, lidocaine, benzocaine, and the like; immunogens (vaccines) for stimulating antibodies against hepatitis, influenza, measles, rubella, tetanus, polio, rabies, and the like; peptides including, but not limited to, leuprolide acetate (an LH-RH agonist), nafarelin, and the like.

Additional substances that can be delivered using hydrogel foams of the invention include hormones, growth factors, and other substances or metabolic precursors capable of promoting growth and survival of cells and tissues or augmenting the functioning of cells, for example, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-alpha), transforming growth factor-beta (TGF-beta), epidermal growth factor (EGF), fibroblast growth factor (FGF), interleukin-1 (IL-1), vascular endothelial growth factor (VEGF) and keratinocyte growth factor (KGF), dried bone material, and the like; and antineoplastic agents such as methotrexate, 5-fluorouracil, adriamycin, vinblastine, cisplatin, tumor-specific antibodies conjugated to toxins, tumor necrosis factor, and the like.

Other useful substances include hormones such as progesterone, testosterone, and follicle stimulating hormone (FSH) (birth control, fertility-enhancement), insulin, and the like; antihistamines such as diphenhydramine, and the like; cardiovascular agents such as papaverine, streptokinase and the like; anti-ulcer agents such as isopropamide iodide, and the like; bronchodilators such as metaproternal sulfate, aminophylline, and the like; vasodilators such as theophylline, niacin, minoxidil, and the like; central nervous system agents such as tranquilizer, B-adrenergic blocking agent, dopamine, and the like; antipsychotic agents such as risperidone, narcotic antagonists such as naltrexone, naloxone, buprenorphine; and other like substances.

The therapeutic agent may be a cell. Examples of cellular agents that may be used with the hydrogels disclosed infra include autologous cells, heterologous cells e.g. cells derived in vitro from stem cells. Stem cells include embryonic stem cells, induced pluripotent stem cells, adult stem cells such as mesenchymal stem cells and adipose derived stem cells. Cells derived from stem cells include cellular progenitor cells (i.e. cells that have not completely matured into an adult phenotype) and fully developed mature adult cells. Cell types include cells of endodermal origin, ectodermal origin and mesodermal origin. Specific cell types may include, but are not limited to cells of the central nervous system such as neurons including dopaminergic neurons, glial cells and astrocytes, cells of the digestive system such as adipose cells, hepatocytes, pancreatic cells including β-islet cells, retinal pigmented epithelial cells, adipose cells and the like.

Where the therapeutic agent is a cell, the cell may attach to the gelatin portion of the hydrogel. Alternatively the cell may be attached to a functionalized monomer within the hydrogel, such as peptide functionalized monomer. Suitable peptides may comprise the RGD sequence. The monomer may be CMHA-S.

The hydrogel foams described infra may be implanted in a subject requiring treatment. For example the hydrogel foams may be implanted into the site of a wound to promote wound healing. The hydrogel foams may be used cosmetically to alter a cosmetic defect. For example the hydrogel foams may be used to treat wrinkles or skin defects or facial wasting associated with HIV. In some instances the hydrogel foams may have one or more cells encapsulated within the hydrogel foam, such as an adipocyte cell. The hydrogel foams of the invention may be administered in the eye to treat corneal epithelial wound healing, to provide corneal tissue engineering, and to provide retinal repair. The hydrogel foams of the invention can be implanted in the central nervous system of a subject to treat conditions such as stroke, Parkinson's disease, spinal cord injury, MS and the like. The hydrogels can be implanted in the digestive tract or under the kidney capsule to treat conditions such as diabetes or liver disease. The hydrogels can be implanted in the heart to treat conditions such as heart failure, arrhythmia and the like.

### Pharmaceutical Compositions and Modes of Administration

The hydrogel foams of the invention may be formulated as a pharmaceutical composition and administered to a subject in need of treatment.

Modes of administration for a therapeutic (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Hydrogel foams as described herein may be administered as a three dimensional construct or as film. The thickness of the hydrogel foam may be adapted to the condition being treated. In some embodiments the components of the hydrogel foam may be combined ex vivo, allowed to polymerize, formed into a foam as described infra and then administered to the subject as a pre-formed hydrogel foam.

The hydrogel foam of the present disclosure can be formulated as a depot preparation. Such long acting formulations can be administered by implantation (for example subcutaneously or intramuscularly) or by intramuscular injection. In some embodiments the composition may be administered by intra-peritoneal injection. In some embodiments the therapeutic disclosed herein can be administered using a catheter.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present disclosure, for example, can be applied to a plaster, or can be applied by transdermal, therapeutic systems that are consequently supplied to the subject.

Specific modes of administration will depend on the indication. The selection of the specific route of administration and the dose regimen is to be adjusted or titrated by the clinician according to methods known to the clinician in order to obtain the optimal clinical response. The amount of therapeutic to be administered is that amount which is therapeutically effective. The dosage to be administered will depend on the characteristics of the subject being treated, e.g., the particular subject treated, age, weight, health, types of concurrent treatment, if any, and frequency of treatments, and can be easily determined by one of skill in the art (e.g., by the clinician).

Pharmaceutical formulations comprising the hydrogel foam of the present disclosure and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present disclosure can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining the therapeutic with pharmaceutically acceptable carriers well known in the art. Such carriers enable the therapeutic of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active therapeutic doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active therapeutic can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the pharmaceutical compositions can take the form of, e.g., tablets or lozenges formulated in a conventional manner.

For administration by inhalation, the therapeutic for use according to the present disclosure is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the therapeutic and a suitable powder base such as lactose or starch.

The compositions of the present disclosure can also be formulated in rectal compositions such as suppositories or retention enemas, e.g., containing conventional suppository bases such as cocoa butter or other glycerides.

Pharmaceutical compositions can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present disclosure can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component may include one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearylfumarate, fatty acid, fatty alcohol, fatty acid ester, glycerylbehenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### Kits

The instant invention also provides kits. The kit may comprise one more monomers suitable for forming a hydrogel. Examples of suitable monomers include hyaluronate, gelatin and heparin. The monomers may be thiolated. The monomers may be carboxymethylated. The monomers may be both thiolated and carboxymethylated. The monomers may be provided in solution, e.g. in a suitable buffer such as PBS or the monomers may be provided as a lyophilized powder. The kit may further include a crosslinking agent e.g. an acrylate. Suitable acrylates include polyethylene glycol dimethylacrylate. The crosslinking agent may be provided in a suitable buffer, e.g. PBS or deionized water. Alternatively, the crosslinking agent may be provided as a lyophilized powder. The kit may further comprise one or more devices for conditioning a hydrogel. For example, the kit may comprise a plurality of syringes. The kit may comprise one or more luer locks, such as plurality of female luer locks. The kit may include one or more vessels suitable for casting a hydrogel. For example the kit may comprise one or more multiwall plates, or one or more petri dishes. The kit may comprise a plurality of plates used to compress the hydrogel foam. The plates may be made of any metal or of glass. The kit may comprise one or more buffers, e.g. PBS or any commercially available cell culture media. The kit may comprise one or more containers. The kit may further comprise one more cells or cell lines suitable for culture on the hydrogel foams. It is contemplated that any of the kit components may be considered optional.

### EXAMPLES

The following examples are not intended to limit the scope of what the inventors regard as their invention nor are they intended to represent that the experiments below are all or the only experiments performed.

### Example 1: Preparation of Hydrogel Foams

Glycosil® (thiol-modified hyaluronate) (BioTime Inc., Alameda, Calif.) and Gelin-S® (thiol-modified gelatin) (BioTime Inc., Alameda, Calif.) and Extralink® (polyethylene glycol diacrylate) (BioTime Inc., Alameda, Calif.) were reconstituted and combined as prescribed by manufacturer's instructions. Briefly, Glycosil®, Gelin-S®, Extralink® and deionized degassed water (DG water) were equilibrated to room temperature. One mL of DG water was aseptically added to each of the tubes containing the Glycosil® and Gelin® and placed on a rocker for about 40 minutes or until the solution was clear and slightly viscous. A half mL of DG water was added to the tube containing the Extralink®. The tube was inverted several times to dissolve the Extralink®. Equal volumes of Glycosil® and Gelin® were mixed together. Extralink® was then added at a 1:4 volume ratio. The resulting mixture was taken into a 35 ml luer lock syringe (Covedian, Mansfield, Mass.) and a second 35 ml luer lock syringe was attached using a female-female luer lock connector (Smiths Medical, Dublin, Ohio). The hydrogel was conditioned by passing the solution between syringes to break apart the macrostructure. Desired consistency is homogenous, thick, flowable and opaque. Opaque material indicates it has been aerated in a uniform manner. This conditioning took place every hour for first few hours, and 24 hours after mixing of components (after complete gelation).

Once complete gelation occurred (˜24 hours after combination of components) conditioning resumed. Conditioned hydrogels were cast as described below. Immediately prior to casting, the conditioned hydrogel was subjected to vigorous conditioning. The conditioned hydrogel was then transferred to a flat bottomed disposable base mold having demensions of 24 mm×24 mm×5 mm (Simport Scientific, Beloeil, Canada). Additionally the syringe used to condition the hydrogel was also used as a casting vessel.

After the conditioned hydrogel was cast into the mold, it was moved to a −20° C. freezer for 18 hours until frozen. A lyophilizer was equilibrated with regard to temperature and pressure while the samples were in the freezer. A tray-style lyophilizer (Labconco, Kansas City, Mo.) was used with a collector temperature of −51° C. and a pressure of 0.030 bar. After lyophilization there a small shrinkage (about 10%) was observed in the conditioned hydrogel.

After complete lyophilization, the hydrogel foam appeared as a white foam-like material that could be easily removed from the mold. The hydrogel foam was then placed between two flat plates and compressed to a thickness of about 1.3 mm and about 0.38 mm to about 0.16 mm until the hydrogel foam formed as a thin film. Uncompressed the conditioned hydrogel had a thickness of about 3.5 mm.

In some experiments, polyvinyl alchol (PVA), a water-soluble synthetic polymer, was added to the gels immediately after the conditioning step. PVA (15% stock in PBS) was mixed with the conditioned gel to a final concentration ranging from 7.5%-0.1% PVA. The PVA-conditioned gel mixture was cast and lyophilized as described supra and gel foam physical properties were inspected after lyophilization and again after rehydration. Addition of 7.5% PVA resulted in a very rigid foam that disintegrated after rehydration. Addition of 2.5% PVA resulted in the formation of a gel foam with characteristics similar to no-PVA gel foams pre-hydration, and a very soft gel foam after rehydration. Gel foams with 1.5% or less PVA had physical characteristics similar to the gel foams with no PVA.

### Example 2: Free Swell Absorptive Capacity of Hydrogel Foams

Hydrogel foams were prepared as described in Example 1. Two formulations of hydrogel foams were tested. Formulation α consisted of 1:1:2 thiolated HA:thiolated gelatin:PEGDA w/w and 2:2:1 v/v. Formulation β consisted of 2:2:1 thiolated HA:thiolated gelatin:PEGDA w/w, and 2:2:1 v/v. The hydrogel foams were cut into samples of about 11 mm×20 mm and allowed to swell by placing the hydrogel foams in deionized water containing 142 mmol sodium ions and 2.5 mmol calcium ions as choloride salts (Sigma-Alrich) for about 2 hours. Samples were weighed both before and after swelling. The results are presented below in Tables 1-3:

TABLE 1

| | | Initial dry | Final wet | Fluid retained |
|---|---|---|---|---|
| Sample ID | Formulation | weight (mg) | weight (mg) | (mg) |
| 1 | α | 25.3 | 334 | 308.7 |
| 2 | β | 25.5 | 200 | 174.5 |
| 3 | β | 24.9 | 207 | 182.1 |
| 4 | α | 23.5 | 320 | 296.5 |

TABLE 2

| | | | | Average |
|---|---|---|---|---|
| | | Gram of | Average | solution |
| | | solution | solution | retained |
| | | retained/g | retained/g of | for each |
| Sample ID | Formulation | of sample (g) | sample (g) | formulation (g) |
| 1 | α | 12.20158103 | 9.743748143 | 12.40930115 |
| 2 | β | 6.843137255 | | |
| 3 | β | 7.313253012 | | 7.078195133 |
| 4 | α | 12.61702128 | | |

TABLE 3

| | | | Average fluid retained |
|---|---|---|---|
| | | Fluid retained | per 100 cm² for each |
| Sample ID | Formulation | per 100 cm² (g) | formulation (g) |
| 1 | α | 14.030415 | 13.75317 |
| 2 | β | 7.931025 | |
| 3 | β | 8.276445 | 8.103735 |
| 4 | α | 13.475925 | |

### Example 3: Cell Growth on Hydrogel Foams

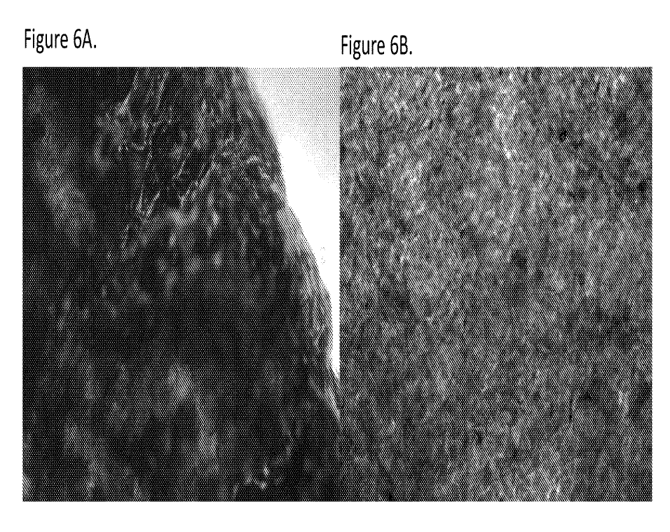

Hydrogel foams of α and β formulations, approximately 5 mm of thickness prior to compression, were prepared according to Example 1, washed in 70% ethanol followed by three rinses in 1×PBS and then suspended in cell culture media (DMEM+10% FBS+1% GlutaMAX™+1% Penicillin/Streptomycin). The hydrogel foams were cut into equal sized pieces; one piece was placed in each well of a 12 well suspension culture plates (Greiner Bio-One). Human mesenchymal stem cells (hMSC) were seeded at a density of 90,000 cells/well. The cells were cultured from 3-10 days, with the cell culture media changed every 48 hours. Cells were stained with Calcein AM (LifeTechnologies) to measure cell viability and Propidium iodide (Sigma-Aldrich, St Louis, Mo.) to stain for necrotic or apoptotic cells. The results showed that the cells attached to the surface of the hydrogel foam and also infiltrated and attached to the interior of the foam. The cells proliferated and remained viable through 10 days of cell culture and the hydrogel foams remained intact (FIG. 6, panels A and B).

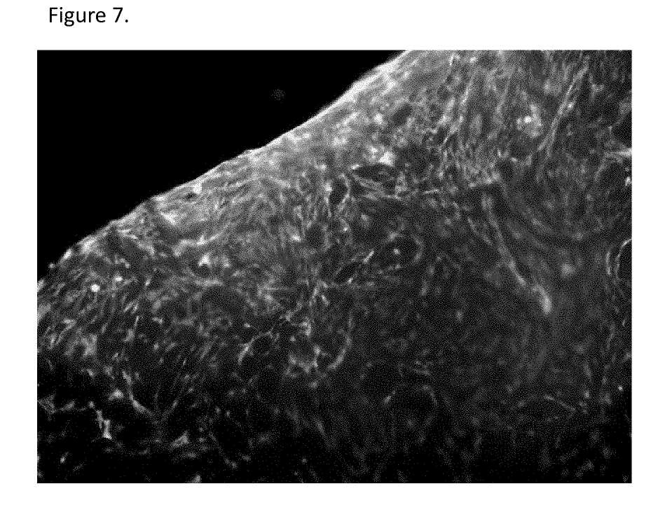

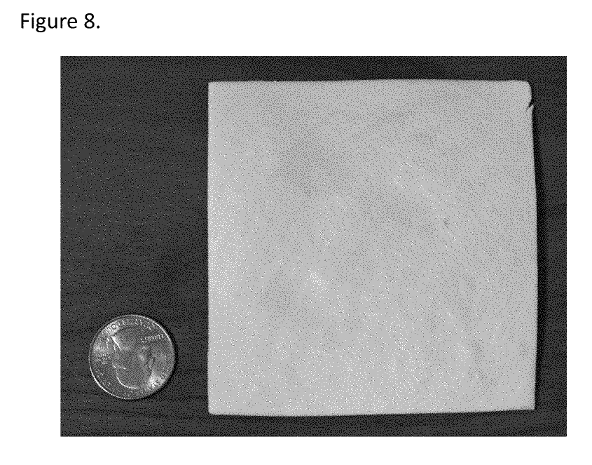

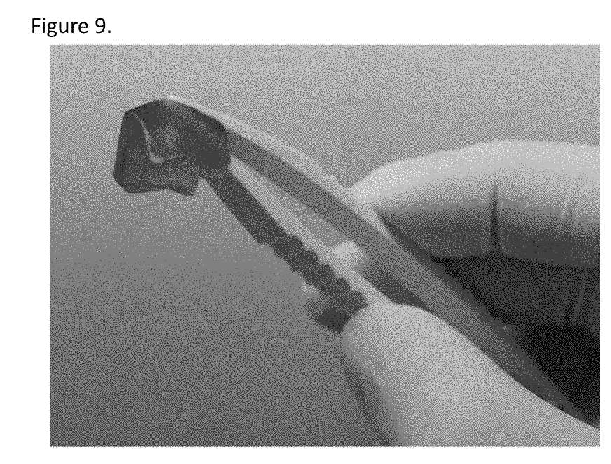

Live/dead imaging (i.e. Calcein AM/propidium iodine staining) of a formulation foam was performed after 10 days of culture. The cells remained viable with the majority of cells stanining with Calcein AM and very few cells staining with propidium iodine (FIG. 7).

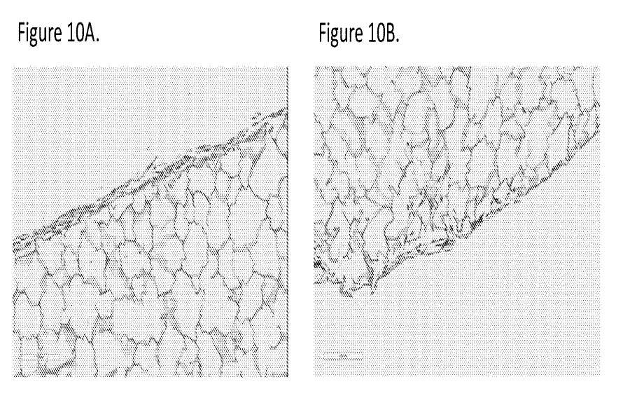

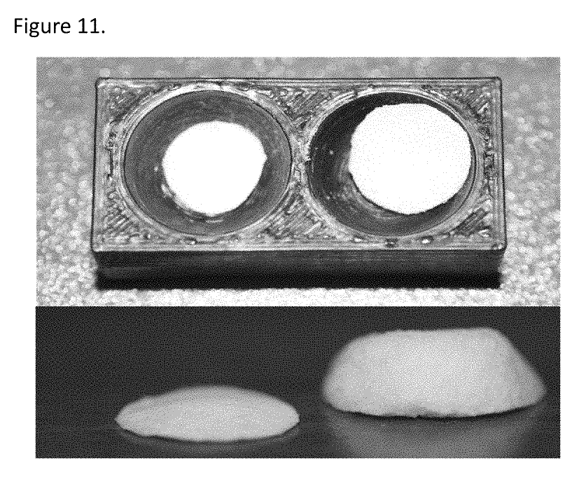

While some cells infiltrated into the interior of the gel foams in the example supra, the infiltration was relatively minor and occurred at breached gel pores (FIG. 10).

The present invention can also be practiced with gel foams where the gel foams have been perforated on purpose to allow for complete infiltration of the gel foam by the cultured cells. Perforation may be done with single needles or by a tool containing a large number of needles or similar tools suitable for achieving partial or complete perforation throughout the entire or partial thickness of the gel foam. The perforation can be done after lyophilization; alternatively, a tool can be incorporated into the liquid hydrogel slurry prior to freezing and removed after lyophilization.

1. A hydrogel foam comprising hyaluronate.

2. The hydrogel foam of claim 1, wherein the hyaluronate is thiolated.

3. The hydrogel foam of claim 1, wherein the hyaluronate is carboxy-methylated.

4. The hydrogel foam of claim 1, wherein the hyaluronate is thiolated and carboxy-methylated.

5. The hydrogel foam of claim 1 further comprising a cross-linker.

6. The hydrogel foam of claim 5, wherein the cross-linker is polyethylene glycol dimethylacrylate.

7. The hydrogel foam of claim 1 further comprising gelatin.

8. The hydrogel foam of claim 7, wherein the gelatin is thiolated.

9. The hydrogel foam of 1, further comprising heparin.

10. The hydrogel foam of claim 1, wherein the hyaluronate is functionalized with a peptide or a nucleic acid.

11. The hydrogel foam of claim 1, wherein the hyaluronate is functionalized with a peptide.

12. The hydrogel foam of claim 11, wherein the peptide is an RGD peptide.

13. The hydrogel foam of claim 1 further comprising a therapeutic agent.

14. The hydrogel foam of claim 13, wherein the therapeutic agent is chosen from a cell, a peptide, a nucleic acid and a small molecule.

15-17. (canceled)

18. A method of making a hydrogel foam, comprising: a) combining at least one polymer and at least one cross-linking agent in a device comprising at least one pore; b) applying an external force to the device from step a) such that the at least one polymer and the at least one cross-linking agent pass through the pore while allowing the at least one polymer and the at least one cross-linking agent to polymerize and form a hydrogel; c) casting the hydrogel from step b) into a vessel; d) freezing the cast hydrogel from step c); and e) lyophilizing the frozen hydrogel from step d), thereby making a hydrogel foam.

19. The method of claim 18, further comprising compressing the hydrogel foam.

20. The method of claim 18, wherein the device is a syringe.

21. The method of claim 18, wherein the force is applied by a human hand.

22. The method of claim 18, wherein the hydrogel is cast in a vessel chosen from a multi-well plate and a petri dish.

23-26. (canceled)

27. The method of claim 19, wherein the hydrogel foam is compressed to a thickness of about 1.3 mm.

28-29. (canceled)
